(19) United States
(12) Patent Application Publication
(10) Pub. No.: US 2026/0331446 A1
(43) Pub. Date: Oct. 22, 2026

(54) METHODS FOR EMPLOYING MASS SPECTROMETRY TECHNIQUES TO SEQUENCE AND ANALYZE BIOLOGICAL MOLECULES

(71) Applicant: PURDUE RESEARCH FOUNDATION, West Lafayette, IN (US)

(72) Inventors: Robert Graham Cooks, West Lafayette, IN (US); Dylan T. Holden, Lafayette, IN (US); Myphuong T. Le, West Lafayette, IN (US); Jeremy Manheim, Kenilworth, NJ (US); Kiran Iyer, Kenilworth, NJ (US)

(21) Appl. No.: 19/108,871

(22) PCT Filed: Aug. 30, 2023
(86) PCT No.: PCT/US2023/031494

§ 371 Date: Mar. 5, 2025

Related U.S. Application Data
(60) Provisional application No. 63/403,984, filed on Sep. 6, 2022.

(51) Int. Cl. H01J 49/00; G01N 33/68; H01J 49/42

(52) U.S. Cl. CPC H01J 49/0036; G01N 33/6818; G01N 33/6848; H01J 49/004; H01J 49/427; G01N 2333/315; G01N 2400/10

(57) ABSTRACT

The invention generally relates to methods for employing mass spectrometry techniques to sequence and analyze biological molecules, such as glycan. In certain aspects, the invention provides methods for sequencing a biological molecule that involve conducting two-dimensional tandem mass spectrometry (2D MS/MS) and in-source collision-induced dissociation (IS-CID) on an ionized biological molecule to generate and analyze a plurality of precursor-product ion pairs of the biological molecule to thereby determine an interrelationship of the plurality of precursor-product ion pairs and sequence the biological molecule.

### RELATED APPLICATION

The present application claims the benefit of and priority to U.S. provisional patent application Ser. No. 63/403,984, filed Sep. 6, 2022, the content of which is incorporated by reference herein in its entirety.

### FIELD OF THE INVENTION

The invention generally relates to methods for employing mass spectrometry techniques to sequence and analyze biological molecules.

### BACKGROUND

Glycans serve crucial roles in both maintaining health and in the progression of disease in addition to possessing major implications for medicine and biotechnology. The biological functions of polysaccharides range from providing structural support to cells, to energy storage, as well as to mediating cellular signaling through post-translational glycosylation of proteins and lipids. Additionally, many vaccines including those for pneumococcus, meningococcus, H. influenzae type b, and S. typhi bacteria utilize the external capsular polysaccharide coating of the bacterial species as a recognition element to bolster host immunity. Despite their clinical relevance, the limited capabilities available to rapidly and accurately quantitate and characterize structural features of glycans has hindered research progress. This shortcoming is further exacerbated by many of the inherent chemical and structural features of polysaccharides including the large diversity in average molecular weights, low ionization efficiency, low in vivo concentrations, frequent presence of isomeric monomer units, varying linkage positions, as well as the highly dynamic macro- and micro-heterogeneity due to their formation via enzymatic processes.

Analysis of glycans has historically been performed using nuclear magnetic resonance (NMR) and/or high-performance liquid chromatography coupled with tandem mass spectrometry (HPLC-MS/MS). While each of these techniques possesses merits and shortcomings, many of the complications just noted affect the experimental design used for analysis as well as the data interpretation. Specifically, there are two standout limitations to most analytical workflows for glycans: (i) the need for sample separation and workup (i.e. preconcentration, solvent exchange, enzymatic and/or chemical degradation, derivatization, etc.) and (ii) the reliance on data-dependent acquisition (DDA) or partially data-independent acquisition (DIA). Both features can dramatically limit sample throughput and the latter requires, in the case of MS/MS, knowledge of the nominal mass of the desired precursor ion(s) or the approximate m/z range of precursor ion(s) to subject to further stages of mass analysis. While preparative workflows increase complexity and specificity, DIA strategies have evolved towards untargeted analysis over large m/z ranges by iterating MS/MS analyses of smaller mass windows. This strategy, while effective in many cases, is still limited by the amount of information that can be gathered for each elution (when chromatographic techniques are utilized) and requires nontrivial data analysis techniques to stitch together and interpret the data collected within each m/z window.

Two-dimensional tandem mass spectrometry (2D MS/MS) was originally demonstrated by Gäumann et al. using a Fourier transform ion cyclotron resonance (FT-ICR) mass spectrometer and later improved by O'Connor and colleagues. A version of this complex mixture analysis technique based on the quadrupole ion trap was developed in our lab. This system can generate, detect, and correlate all the product ions formed by fragmentation of all precursor ions generated from a sample in less than one second. Additional information concerning the instrumentation and technical aspects of generating and applying the 2D MS/MS activation and fragmentation waveforms to the ion trap electrodes, as well as insight into data collection can be found in Materials and Methods.

The data resulting from a 2D MS/MS experiment is best displayed on a three-dimensional surface where the precursor m/z and product m/z values lie on the x- and y-axes, respectively, and the abundance of a precursor-product ion pair is denoted by color and/or peak intensity along the z-axis. Each individual feature in a 2D mass spectrum (i.e. a precursor-product ion pair) is equivalent to the output of a single Multiple Reaction Monitoring (MRM) measurement representing the transition from one precursor ion to one product ion. One can extract the equivalent of a product ion scan (i.e. all product ions generated from a single precursor ion) by taking a vertical slice of the 3D spectrum, while extracting a horizontal slice provides the equivalent of a precursor ion scan (i.e. all precursor ions which fragment to a given product ion). Perhaps most importantly, precursor-product ion pairs that fall on a given diagonal of constant slope are related through a shared neutral loss during fragmentation. Species with shared neutral loss values are often related to one another by the presence of specific functional groups. Lastly, the diagonal line along which precursor ion m/z is equal to product ion m/z is deemed the autocorrelation line and represents a version of the full scan mass spectrum, though this information is not usually gathered from a 2D MS/MS experiment.

Besides the speed of analysis, 2D MS/MS differs from other DIA methods in that it conserves (and illustrates) the relationship between precursor-product ion pairs and is not subject to a limit on the size of the precursor m/z range that is analyzed other than those imposed by the geometric and electronic parameters of the mass analyzer. Furthermore, visualization of the data via a 2D mass spectrum decreases the apparent chemical noise. This feature, which is also present when comparing MS/MS data to full scan mass spectra, enables the direct analysis of chemical species from complex matrices and dramatically reduces or even eliminates the need for any separation or purification steps prior to analysis.

Enzymatic or chemical degradation of full-length glycans prior to analysis, while effective in bringing the masses of the precursor ion(s) into a range which is accessible to commercial mass spectrometers, is capable of generating a range of degradant species that may not be readily predictable. Some of these species can be highly insightful in terms of providing information about structure, although they may be too low in abundance to be accessed in DDA methods and their fragments may be too low in intensity to be deemed informative in DIA workflows. While 2D MS/MS can address these shortcomings, the need for degradation of full-length polysaccharides remains a significant limitation on analytical throughput.

### SUMMARY

The invention recognizes that mass spectrometry (MS) is a powerful tool for structural characterization of biological molecules, such as polysaccharides, a key objective in glycomics as well as in vaccine development. Traditional methods of glycan analysis often rely on tandem MS (MS/MS) analysis of smaller, more manageable fragments resulting from enzymatic and/or chemical degradation of full-length biopolymers. However, this approach is time consuming, often involving arduous separation and purification steps, and requires targeted selection of the degradant species to be subjected to MS/MS analysis.

The invention provides an approach that involves the use of two-dimensional tandem mass spectrometry (2D MS/MS), a fully data-independent acquisition technique that allows for the fragmentation of all precursor ions (in less than a second in this realization) and which conserves the relationship between precursor and product ion pairs, to analyze various pneumococcal capsular polysaccharide serotypes directly from buffered solution. By combining this technique with in-source collision-induced dissociation (IS-CID), a function available on nearly all commercial mass spectrometers, we show that it is possible to generate a unique spectral fingerprint for each serotype and to also elucidate the structural connectivity between different precursor-product ion pairs thus effectively sequencing the glycan. Other features, such as the pattern of acylations, can also be deduced from this data.

In certain aspects, the invention provides methods for sequencing a biological molecule that involve conducting two-dimensional tandem mass spectrometry (2D MS/MS) and in-source collision-induced dissociation (IS-CID) on an ionized biological molecule to generate and analyze a plurality of precursor-product ion pairs of the biological molecule to thereby determine an interrelationship of the plurality of precursor-product ion pairs and sequence the biological molecule. In certain embodiments, the ionized biological molecule is a polysaccharide, peptide or glycopeptide. In certain embodiments, the polysaccharide is a glycan. In certain embodiments, the glycan is associated with a bacterium. In certain embodiments, the method further comprises identifying the bacteria based on the sequence of the glycan.

Other aspects of the invention provide, methods for sequencing a pneumococcal polysaccharide that involve conducting two-dimensional tandem mass spectrometry (2D MS/MS) and in-source collision-induced dissociation (IS-CID) on an ionized pneumococcal polysaccharide to generate and analyze a plurality of precursor-product ion pairs of the pneumococcal polysaccharide to thereby determine an interrelationship of the plurality of precursor-product ion pairs and sequence the pneumococcal polysaccharide. In certain embodiments, the pneumococcal polysaccharide is a pneumococcal capsular polysaccharide. In certain embodiments, the method further comprises identifying a pneumococcal based on the sequence of the pneumococcal capsular polysaccharide.

In certain embodiments, an rf trapping voltage is held constant to maintain a secular frequency of trapped ions throughout an entire scan. In certain embodiments, externally generated auxiliary waveforms are then applied, individually but simultaneously, to orthogonal x-and y-rod pairs of an ion trap. In certain embodiments, the waveform applied to the y-rod pairs fragments precursor ions by nonlinearly sweeping through a range of ion secular frequencies. In certain embodiments, nonlinearity of the frequency sweep produces a linear m/z scale in ions subjected to fragmentation with time. In certain embodiments, a second waveform is applied to the x-rods to perform nonlinear frequency sweeps to eject generated product ions into the detector. In certain embodiments, a rate of product ion ejection events is greater than that of precursor m/z fragmentation events and is timed to preserve a relationship of product ions to their respective precursor ions. In certain embodiments, the product ion m/z information is determined by a temporal signal detected at a given product ion ejection event while the precursor m/z information is deduced from a time at which that signal is detected within one full mass scan.

### BRIEF DESCRIPTION OF THE FIGURES

FIG. 1 panel A shows two-dimensional tandem mass spectrometry (2D MS/MS) performed by application of supplementary AC waveforms to the ion trap electrodes of a commercial mass spectrometer. Precursor ions are activated in the y-dimension while product ions are rapidly ejected in the x-dimension towards the detector. A 2D MS/MS spectrum (recorded in a single scan) and representing all the precursor ions and all their respective product ions is typically produced in 700-1500 ms. FIG. 1 panel B shows a hypothetical 2D MS/MS spectrum for a given compound. A single vertical line through the spectrum represents all the product ions that are generated by the fragmentation of a given precursor ion and when extracted it has one mass dimension and represents a product ion scan. Horizontal lines correspond to all the precursor ions that fragment to a given product ion (precursor ion scan). Diagonal neutral loss lines represent precursor-product ion pairs that fragment by the loss of an isobaric neutral moiety, often associated with the presence of the similar functional groups.

FIG. 2 panel A shows a full scan mass spectrum of the Pneumococcal capsular polysaccharide serotype 31F (PnP 31F) in Tris-HCl buffer (pH 6.5) with 10 mM NaCl ionized by negative mode nESI and analyzed using a Thermo LTQ-XL mass spectrometer. The large cluster of peaks observed at higher m/z values corresponds to the presence of multiple charge states, as well as to water and buffer adducts of the full-length polysaccharide and degradation products formed in solution or during the ionization process. FIG. 2 panel B shows a full scan mass spectrum of PnP 31F using similar conditions to those described in A, but with the application of in-source fragmentation (tube lens value increased from −100 V to-250 V). The most abundant peaks in the spectrum correspond to various glycosidic bond cleavages and cross-ring fragmentations of the polysaccharide repeating unit. These source fragments are annotated with proposed ionic structures.

FIG. 3 shows a series of 2D MS/MS spectra recorded using standard ion injection and 2D MS/MS fragmentation (A), the same experiment but with in-source (IS) CID (B), the same experiment but with SWIFT activation but w/o IS-CID (C) and finally the full experiment with IS-CID and SWIFT activiation (D).

FIG. 4 panel A shows a posterior mass spectrum recorded for PnP 31F using the modified 2D MS/MS instrument with a broadband activation waveform for in-source fragmentation and processed by extracting data from the 2D MS/MS data domain along the precursor mass axis (x-axis). This selection corresponds to all the precursor ions in the sample. FIG. 4 panel B shows an observed IS-CID full scan mass spectrum recorded using the standard LTQ mass spectrometer.

FIG. 5 shows an annotated IS-CID-2D-MS/MS spectrum of serotype PnP 31F performed using modified LTQ ion trap mass spectrometer. Stepwise patterns are drawn to sequentially reconstruct the repeating unit. The four patterns diverge from the starting point are exactly 42 Da apart and represent four degrees of acylation, from zero to a maximum of three. The autocorrelation line is shown in yellow.

FIG. 6 is an IS-CID-2D-MS/MS spectra of a mixture containing four Pneumococcal polysaccharide serotypes: PnP 3V, PnP8, PnP22F, and PnP31.

FIG. 7 is a schematic providing an overview of certain methods of the invention.

FIG. 8 is an IS-CID 2D MS/MS spectrum of leucine-enkephalin (YGGFL) collected in the positive ion mode.

FIG. 9 is an illustration showing an exemplary data analysis module for implementing the systems and methods of the invention in certain embodiments.

### DETAILED DESCRIPTION

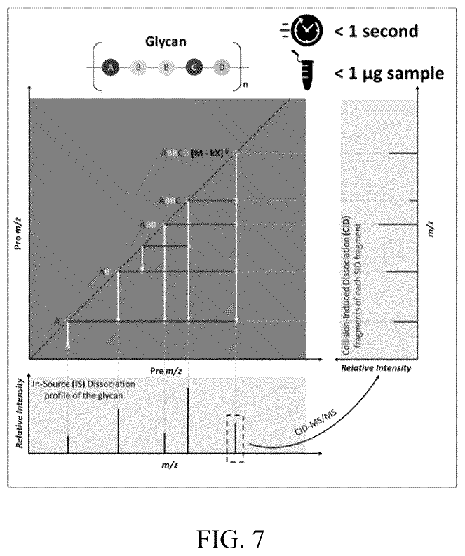

The invention provides an approach that combines 2D MS/MS with in-source collision-induced dissociation (IS-CID), a method that augments the activation/desolvation energy supplied at the interface of the ion source and MS inlet by increasing the value of the tube lens or skimmer voltage relative to the capillary voltage. This effectively perform gas-phase degradation of nano-electrosprayed intact pneumococcal polysaccharides. This process simultaneously removes most salt and water adducts that are typically generated when ionizing directly from buffered solution or biological matrices. Due to the polymeric nature of glycans, patterns in the 2D MS/MS spectra generated from the analysis of the degradant species (in the form of precursor ions) allows one to rebuild the repeating units of the polysaccharide from the bottom-up or top-down when reading the acquired data. See FIG. 7.

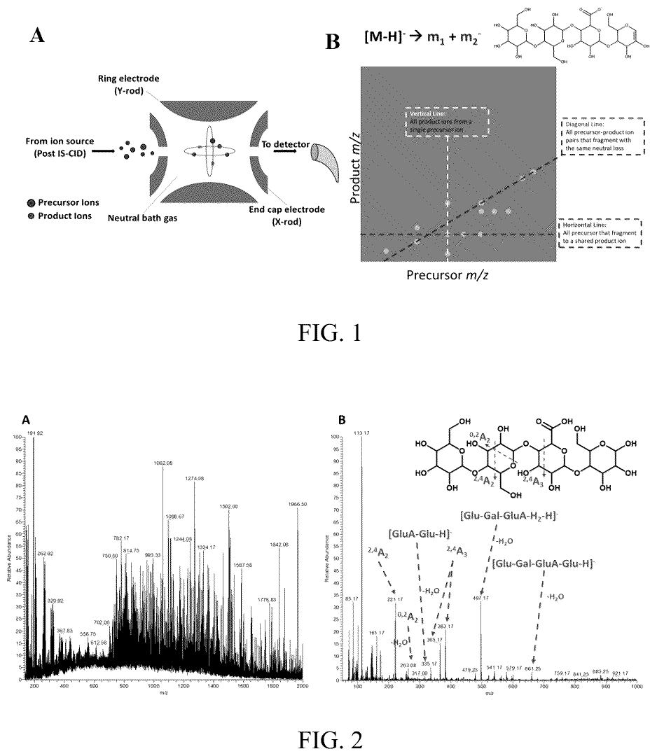

This study initially sought to develop a high throughput method for analysis of pneumococcal polysaccharides that (i) required minimal sample preparation, (ii) was compatible with ambient ionization methods, and (iii) could be carried out using a commercial mass spectrometer. Linear ion trap nESI-MS analysis of full-length pneumococcal polysaccharides which were reconstituted in 10 mM Tris buffer resulted in broad clusters of peaks hundreds of m/z values in width consisting of various charge states and salt adducts of the presumably intact compound (FIG. 2 panel A). Many unsuccessful attempts were made to optimize on-line desalting procedures (i.e. increased inlet temperature, electrophoretic cleanup^{36,37}, paper spray ionization³⁸) that would provide more informative spectra. It was observed, however, that in-source fragmentation (IS-CID), by imparting enough additional energy into the intact polysaccharide to cause fragmentation, almost eliminated the broad adduct cluster and revealed peaks of significant intensity at lower m/z values, many of which were spaced apart at regular mass intervals. In source fragmentation is not often used as it can lead to mis-annotation of product ions given the absence of information on precursor ions.^{39,40} However, in this case the IS-CID peaks (FIG. 2 panel B) corresponded to predictable glycosidic bond cleavages and cross-ring fragmentation products of the polysaccharide repeating units. Clearly the energy imparted into the full-length polysaccharide was sufficient to produce a range of relatively low mass and structurally informative fragments.

It should be possible to isolate and subject each of the source fragment ions to IT-CID and in that way deduce the relationship of the conventional CID fragment ions and the in-source fragments to each other without being limited by low signal intensity during later rounds of MSⁿ as is commonly an issue during top-down analysis. This approach is data-dependent, which increases the chances for human error and it would take the user a considerable amount of time to individually isolate, fragment, and infer the relationships between the species of interest to confidently determine the sequence polysaccharide repeating unit(s).

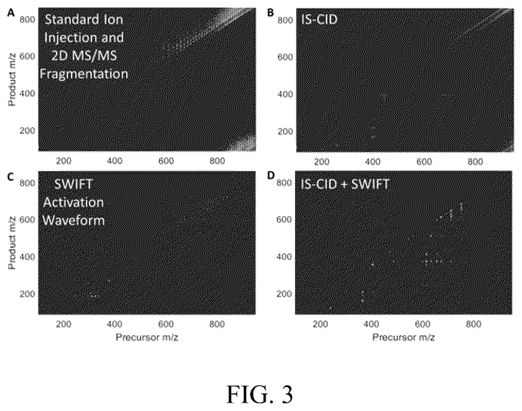

Given these limitations, 2D MS/MS was employed to carry out data-independent analysis and eliminate the need for manual MS/MS analysis of the observed in-source fragments. It was found that source fragmentation was not as simple to perform using the modified MS used for 2D MS/MS as compared to the commercial MS. This is likely because the 2D MS/MS instrument uses N₂ bath gas (to increase sensitivity) rather than He as was the case for the previous experiments. This difference directly affects the conditions in the interfacial region responsible for in-source fragmentation. From FIG. 3 panel A one can observe that the 2D MS/MS spectrum of pneumococcal polysaccharide 31 (PnP 31) without the application of in-source fragmentation features a large packet of signals at high mass on the observed autocorrelation line, a feature which is often attributed to unfragmented precursor ions. After adjusting the ion optical parameters to favor source fragmentation (i.e. increasing the tube lens voltage DC offset) the 2D MS/MS spectrum of PnP8 featured slightly less of the unfragmented precursor ion signals and exhibited faint spectral features at precursor and product m/z values that fall on similar horizontal or vertical lines, results which correspond to low-efficiency IS-CID processes (FIG. 3 panel B).

Custom waveforms are normally applied to the x-and y-rods in the modified 2D MS/MS instrument through an external waveform generator to facilitate ion trapping in addition to simultaneous precursor ion activation and product ion ejection. It is possible, however, to apply additional waveforms to the ion trap to carry out other scanning functions. To accommodate the poor performance of IS-CID observed on this instrument, a stored waveform inverse Fourier transform (SWIFT) excitation waveform of low amplitude and broad frequency range was applied to the ion trap to fragment ions entering the ion trap such as the intact polysaccharide, without ejecting the resulting fragments. This process effectively increases the abundance of IS-CID fragments while adding only a few hundred milliseconds of measurement time. Once this process was complete the standard 2D MS/MS scan waveforms were then applied to fragment all of resulting IS-CID species. When only the SWIFT activation waveform is applied the 2D MS/MS spectra for each of the serotypes revealed an ordered pattern of low-intensity spectral features and a lower amount of unfragmented precursor ion signals (FIG. 3 panel C). However, when IS-CID was applied in conjunction with the SWIFT waveform, the unfragmented precursor ion signal was completely absent from the 2D spectrum and the intensity of the precursor-product ion spectral features was increased compared to the SWIFT-only case (FIG. 3 panel D).

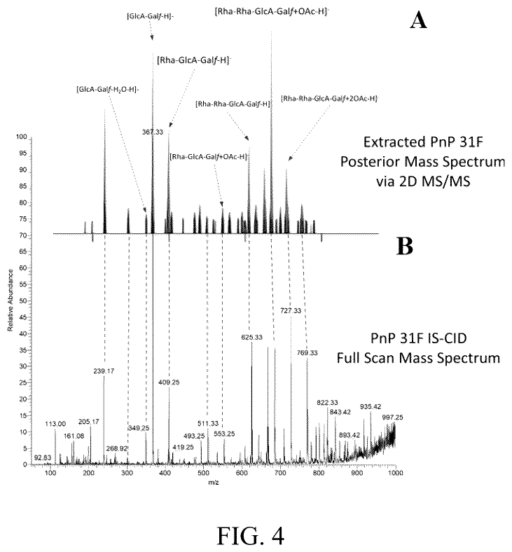

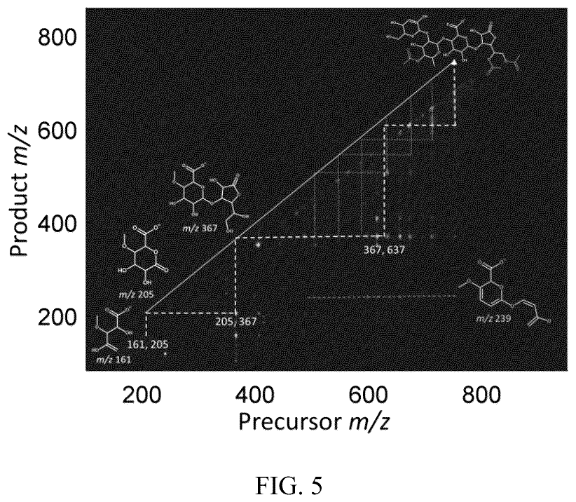

The precursor-product ion pairs observed in the 2D MS/MS spectra for each of the polysaccharides agreed well with the IS-CID ions observed using a conventional mass spectrometer with source dissociation. This showed that the 2D MS/MS methodology was not only capable of producing the same species as observed on a completely different instrument in a fraction of the time but also produced additional, less common cross-ring fragments (FIG. 4 panels A-B). Cross-ring fragments are not as accessible as glycosidic bond cleavages during low energy collision processes, but they can provide useful information on the sites of chemical modifications (e.g. N-acylation) and bond linkages.

The patterns of the spectral features resulting from in-source CID combined with 2D MS/MS on the pneumococcal polysaccharides contains the sets of relationships typically observed in 2D MS/MS spectra: product ion scan lines (vertical), precursor ion scan lines (horizontal), neural loss scan lines (diagonal). However, due to the polymeric nature of the oligosaccharides combined with both in-source and IT-CID fragment ion generation, a stepwise or triangular pattern of connections was observed between many precursor-product ion pairs. As described previously for IS-CID and MS/MS performed on the standard commercial mass spectrometer, many of the product ions resulting from the dissociation of in-source fragments were identical in m/z to other lower mass source fragments. That is, the same fragments were being generated by two different dissociation methods. While these connections could be deduced by fragmenting the individual source fragments and determining which source fragments were related to each other via shared precursor and product ions, 2D MS/MS inherently retains these familial relationships.

The stepwise pattern in a two-dimensional mass spectrum is realized when the product ion of a precursor-product ion pair (i.e. a single spectral feature) and the precursor ion of another precursor-product ion pair coincide. By starting at any given feature in the spectrum one can travel vertically towards the autocorrelation line (where precursor m/z =product m/z) or horizontally also to higher mass (right) until another feature is found on this line. If this holds, the two species satisfy the precursor and product ion relationship mentioned above and can be said to be related in structure (i.e. the lower mass precursor-product ion pair can be formed by fragmentation of the higher mass precursor-product ion pair). The stepwise pattern can be repeated until no additional precursor-product ion pairs satisfy the shared m/z requirement or until the end of m/z range in either dimension of the spectrum is reached. These relationships enable one to understand the structural connectivity of fragments. For example, the lowest mass product ion must come from a certain precursor ion(s) that falls on the stepwise pattern and not from others that do not. That precursor then serves as the product ion for another higher mass precursor ion and so on, effectively rebuilding the polymer units from any given point and moving up (or down) in m/z. It is impossible to determine the spatial connectivity of the fragment ions within a precursor from a single product ion scan alone though this information may be accessible from MSⁿ experiments. The general scheme for this process is best represented in FIG. 5 along with the same process repeated for the 2D spectrum of PnP 31.

To explore the utility of this technique, four intact pneumococcal polysaccharide serotypes (PnPs 3, 8, 22F, 31) with average molecular weights ranging from 20-80 kDa and known repeating unit structures were ionized directly from buffer and consecutively fragmented by IS-CID 2D MS/MS to each yield a unique spectral fingerprint. From the resulting spectra, it was possible to start from a given precursor-product ion pair and follow the stepwise pattern to rebuild the repeating unit structure. Though the repeating units were generally known beforehand, this approach allowed for determination of the exact locations for the three acetylation modifications possible on PnP 31, a topic on which there has been some uncertainly. To assess the generality of the method, the same process was carried out for two non-pneumococcal capsular polysaccharides, hyaluronic acid and alginic acid, for which the same phenomenon was observed as with the four serotypes.

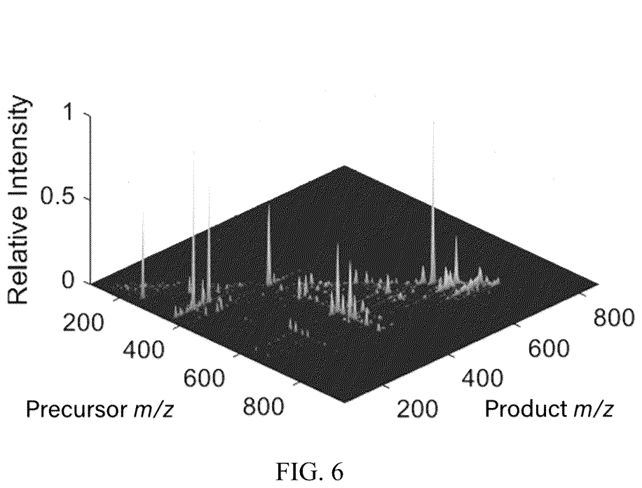

In an effort to simulate a simple vaccine product, the four buffered pneumococcal polysaccharides were combined in equal concentrations and analyzed via IS-CID 2D MS/MS. As seen in FIG. 6, features corresponding to each of the serotypes can be observed in the 2D mass spectrum with minimal loss of information compared to that obtained when each serotype was analyzed individually. Intensities of the product ion pairs can change in the mixture due to expected matrix effects, the different molecular weight of the full-length polysaccharide, as well as fragmentation and ionization efficiency differences in the various glycan components. Despite the large number of spectral features, it is possible still to iteratively follow the stepwise/triangular pattern from any given point and determine that there are four unrelated species in the mixture. The sets of precursor-product ion pairs group together even if one does not know the identity of the analytes. PnPs 8 and 22F with the case of product ion m/z 383.

When mixture components share a common product ion, the product ion can still be differentiated provided that the respective precursor ions are differ in mass. This is the case for PnPs 8 and 22F for the product ion m/z 383.

It has been demonstrated that in-source dissociation in conjunction with 2D MS/MS is capable of both the gas-phase degradation of full-length pneumococcal capsular polysaccharides and fully data-independent acquisition of the degradant species and their corresponding fragment ions. The resulting data is shown to be as at least as informative as when individual product ion scans are performed in a targeted fashion using a traditional mass spectrometer, while being performed in a fraction of the time. Due to the presence of repeating units in the polysaccharides as well as the consecutive of the fragments generated from IS-CID and IT-CID, it is possible to use the pattern(s) of features in two-dimensional mass spectra to infer the connectivity of precursor-product ion pairs thereby sequencing the repeating units. This pattern follows because many of the precursor ‘degradant’ ions generated during IS-CID are also product ions that from the in-trap dissociation of other, larger precursor ions. Correlating these spectral features enables reconstruction of repeat units and assists in determining the location and nature of branch points, sites and types of chemical modifications, and other molecular features. This approach has also been shown to work in a mixture of four different pneumococcal serotypes, as well as for two other common biologically relevant polysaccharides.

The methodology described here removes or reduces laborious sample preparation steps including purification or chemical and/or enzymatic degradation and performs these functions solely with the mass spectrometer. Scan times are less than one second. While the data presented is meant to demonstrate proof-of-concept for the analysis of polysaccharides with 2D MS/MS, extension to glycoproteins, glycolipids, and other biopolymers is underway. Additional techniques such as on-line derivatization will also be explored in an effort to provide additional structural information (i.e. O- or N-linkages, reducing end determination) and improve ionization efficiency without any significant hindrances to sample throughput. Automated data analysis workflows are currently being developed to allow sequence elucidation of polysaccharides with unknown repeating unit structures.

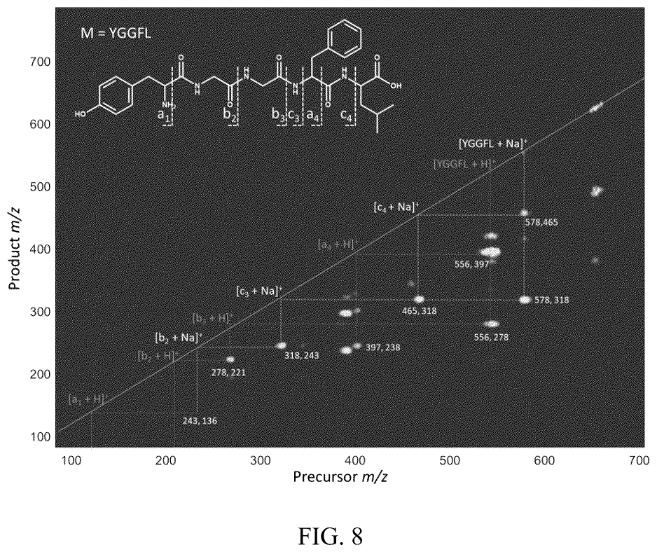

FIG. 8 is an IS-CID 2D MS/MS spectrum of leucine-enkephalin (YGGFL) collected in the positive ion mode. The peptide was dissolved in a 1:1 mixture of water and methanol at a concentration of 50 micromolar (μM) and ionized using nano-electrospray ionization (nESI). In-source collision-induced dissociation (IS-CID) was accomplished through the application of source activation energy (18 V) via the Thermo LTQ Tune instrument software and was further augmented by a 50-millisecond (ms) stored waveform inverse Fourier transform (SWIFT) activation waveform (2.9 V) applied to the Y-rods of the ion trap via an external waveform generator and summing amplifier. This process was implemented previously for the IS-CID-induced degradation of full-length pneumococcal capsular polysaccharides. However, in this case, the activation waveform utilized in the 2D MS/MS experiment to increase the abundance and type of fragment ions following IS-CID was applied at a significantly lower amplitude than in the case of the polysaccharides (400 mV vs. 1.7 V) as it found to contribute to a substantial loss in overall signal intensity. Even without the application of the 2D MS/MS activation waveform, the IS-CID methodology described above was sufficient in producing a broad distribution of abundant multi-generational fragment species prior to mass analysis. The scan length of the entire IS-CID 2D MS/MS process was 950 ms (50 ms for SWIFT, 900 ms for the 2D MS/MS ejection waveform) and the spectrum shown is the average of 20 individual spectra.

Stepwise patterns which inflect at the autocorrelation line (where precursor ion m/z is equal to product ion m/z) can be observed between many of the precursor-product ion pair spectral features, though only two are shown in this case. This is made possible by the creation of at least two generations of fragment species from the protonated analyte ion followed by 2D MS/MS analysis. Given that some of the ions in the first generation of fragment species match nominally in product m/z to the precursor m/z of some of the second generation of fragment species, one can then connect features that meet the above criterion to determine the structural connectivity of the fragment ions (i.e. sequencing the peptide analyte). The species which fall upon the selected stepwise pattern shown above match well with the fragments observed both from IS-CID full scan mass spectra and with product ion spectra (up to MS5) of the protonated or sodiated YGGFL analyte on a traditional LTQ mass spectrometer. Precursor-product ion pairs are annotated using the peptide sequence tag notation first introduced by Roepstorff and Fohlman (Biomed. Mass Spectrom. 1984, 11 (11 ): 601. doi:10.1002/bms.1200111109) which is considered the gold standard in the proteomics community.

### System Architecture

In certain embodiments, the systems and methods of the invention can be carried out using automated systems and computing devices. Specifically, aspects of the invention described herein can be performed using any type of computing device, such as a computer, that includes a processor, e.g., a central processing unit, or any combination of computing devices where each device performs at least part of the process or method. In some embodiments, systems and methods described herein may be controlled using a handheld device, e.g., a smart tablet, or a smart phone, or a specialty device produced for the system.

Systems and methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

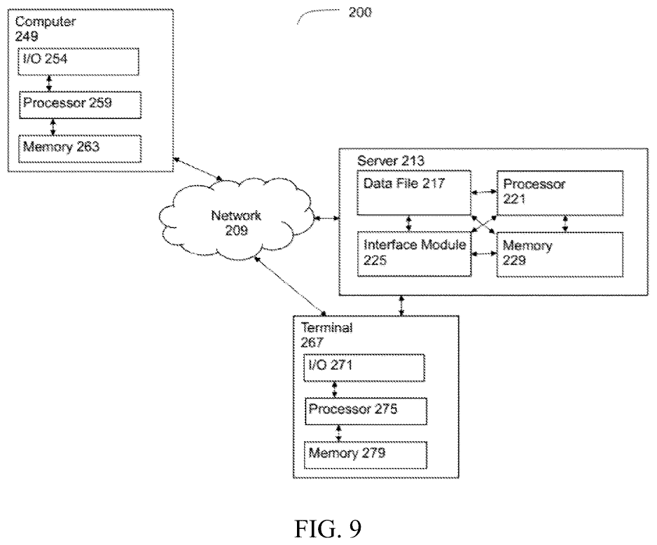

In an exemplary embodiment shown in FIG. 9, system 200 can include a computer 249 (e.g., laptop, desktop, or tablet). The computer 249 may be configured to communicate across a network 209. Computer 249 includes one or more processor 259 and memory 263 as well as an input/output mechanism 254. Where methods of the invention employ a client/server architecture, steps of methods of the invention may be performed using server 213, which includes one or more of processor 221 and memory 229, capable of obtaining data, instructions, etc., or providing results via interface module 225 or providing results as a file 217. Server 213 may be engaged over network 209 through computer 249 or terminal 267, or server 213 may be directly connected to terminal 267, including one or more processor 275 and memory 279, as well as input/output mechanism 271.

System 200 or machines according to the invention may further include, for any of I/O 249, 237, or 271 a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory 263, 279, or 229 according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein

### EXAMPLES

### Example 1: Materials and Methods

### Chemicals

Pneumococcal polysaccharides (US-designation serotypes (PnP 3, 8, 22F, 31) were purchased from ATCC (Manassas, VA, USA) while PnP 31F was provided by Merck & Co. (Rahway, NJ, USA). Sodium alginate and hyaluronic acid were used as experimental standards and were purchased from Sigma-Aldrich (St. Louis, MO, USA). All polysaccharides were reconstituted in water or 1 M Tris-HCl (Sigma Aldrich) at pH 6.5 and 1 mM NaCl and diluted to a working concentration of 150 parts per million (ppm) (w/v).

### Ionization

Solutions were ionized by nanoelectrospray ionization (nESI) using borosilicate glass capillaries (1.5 mm o.d., 0.86 mm i.d.) from Sutter Instruments (Novato, CA, USA) which were pulled to a final tip diameter of ca. 5 μm with a Flaming/Brown micropipette puller (model P-97, Sutter Instruments). The electrospray emitter and holder (Warner Instruments, Hamden, CT, USA) was placed approximately 2 cm away from the inlet of the mass spectrometer while a −2 kV potential was applied to initiate electrospray. All spectra were recorded in the negative ion mode.

### Instrumentation

Two instruments were used in this study. One was an unmodified Thermo Finnigan LTQ mass spectrometer (San Jose, CA, USA), which was used to perform in-source dissociation and standard one-dimensional mass spectrometry. It used He as ion trap bath gas at an uncalibrated ion gauge reading of 1.9×10⁻⁵ torr. IS-CID was performed on the unmodified LTQ by using the preset Source Fragmentation functionality on the Thermo instrument tuning software (LTQ Tune).

The second instrument which was used for all 2D MS/MS measurements was a modified Thermo Finnigan LTQ mass spectrometer. It utilized N₂ bath gas at an uncalibrated ion gauge reading of 1.7×10⁻⁵ torr. 2D MS/MS waveforms were generated using two Keysight 33612A arbitrary waveform generators with 64 megasample memory upgrades (Newark element 14, Chicago, IL, USA). Waveforms were defined using MATLAB (Mathworks, Natick, MA, USA), exported as as.csv files, and then imported into the waveform generator software. The 2D MS/MS scans were 900 ms in length and the displayed spectra are the average of 25 individual scans. IS-CID was achieved on the modified 2D MS/MS instrument by increasing the tube lens voltage from −100 V (no IS-CID) to −250 V (IS-CID). When noted, an additional stored waveform inverse Fourier transform (SWIFT) ion excitation waveform was applied to the x-rod pairs of the instrument to assist in producing additional IS-CID product ions prior to their being subjected to further 2D MS/MS fragmentation and analysis. The need for different IS-CID parameters is likely due to the difference in bath gas between the two instruments (Commercial LTQ-He, 2D MS/MS LTQ-N₂). N₂ is used for the 2D MS/MS experiments to improve sensitivity, as demonstrated previously.

As shown in FIG. 1, the 2D MS/MS methodology requires that the rf trapping voltage be held constant to maintain the secular frequency of the trapped ions throughout an entire scan. Externally generated auxiliary waveforms are then applied, individually but simultaneously, to the orthogonal x- and y-rod pairs of the ion trap. The waveform applied to the y-rod pairs fragments the precursor ions by nonlinearly sweeping through a range of ion secular frequencies. The nonlinearity of this frequency sweep produces a linear m/z scale in the ions subjected to fragmentation with time. A second waveform is applied to the x-rods to perform nonlinear frequency sweeps to quickly eject the generated product ions into the detector.²⁶ The rate of product ion ejection events is many times greater than that of the precursor m/z fragmentation events and is timed to preserve the relationship of product ions to their respective precursor ions. The product ion m/z information is determined by the temporal signal detected at a given product ion ejection event while the precursor m/z information is deduced from the time at which that signal is detected within one full mass scan. More detailed descriptions of this 2D MS/MS scan methodology can be found in previous publications.

1. A method for sequencing a biological molecule, the method comprising: conducting two-dimensional tandem mass spectrometry (2D MS/MS) and in-source collision-induced dissociation (IS-CID) on an ionized biological molecule to generate and analyze a plurality of precursor-product ion pairs of the biological molecule to thereby determine an interrelationship of the plurality of precursor-product ion pairs and sequence the biological molecule.

2. The method of claim 1, wherein the ionized biological molecule is a polysaccharide, peptide or glycopeptide.

3. The method of claim 2, wherein the polysaccharide is a glycan.

4. The method of claim 3, wherein the glycan is associated with a bacteria.

5. The method of claim 4, wherein the method further comprises identifying the bacteria based on the sequence of the glycan.

6. The method of claim 1, wherein an rf trapping voltage is held constant to maintain a secular frequency of trapped ions throughout an entire scan.

7. The method of claim 6, wherein externally generated auxiliary waveforms are then applied, individually but simultaneously, to orthogonal x-and y-rod pairs of an ion trap.

8. The method of claim 7, wherein the waveform applied to the y-rod pairs fragments precursor ions by nonlinearly sweeping through a range of ion secular frequencies.

9. The method of claim 8, wherein nonlinearity of the frequency sweep produces a linear m/z scale in ions subjected to fragmentation with time.

10. The method of claim 9, wherein a second waveform is applied to the x-rods to perform nonlinear frequency sweeps to eject generated product ions into the detector.

11. The method of claim 10, wherein a rate of product ion ejection events is greater than that of precursor m/z fragmentation events and is timed to preserve a relationship of product ions to their respective precursor ions.

12. The method of claim 11, wherein the product ion m/z information is determined by a temporal signal detected at a given product ion ejection event while the precursor m/z information is deduced from a time at which that signal is detected within one full mass scan.

13. A method for sequencing a pneumococcal polysaccharide, the method comprising: conducting two-dimensional tandem mass spectrometry (2D MS/MS) and in-source collision-induced dissociation (IS-CID) on an ionized pneumococcal polysaccharide to generate and analyze a plurality of precursor-product ion pairs of the pneumococcal polysaccharide to thereby determine an interrelationship of the plurality of precursor-product ion pairs and sequence the pneumococcal polysaccharide.

14. The method of claim 13, wherein the pneumococcal polysaccharide is a pneumococcal capsular polysaccharide.

15. The method of claim 14, wherein the method further comprises identifying a pneumococcal based on the sequence of the pneumococcal capsular polysaccharide.

16. The method of claim 13, wherein an rf trapping voltage is held constant to maintain a secular frequency of trapped ions throughout an entire scan.

17. The method of claim 16, wherein externally generated auxiliary waveforms are then applied, individually but simultaneously, to orthogonal x-and y-rod pairs of an ion trap.

18. The method of claim 17, wherein the waveform applied to the y-rod pairs fragments precursor ions by nonlinearly sweeping through a range of ion secular frequencies.

19. The method of claim 18, wherein nonlinearity of the frequency sweep produces a linear m/z scale in ions subjected to fragmentation with time.

20. The method of claim 19, wherein a second waveform is applied to the x-rods to perform nonlinear frequency sweeps to eject generated product ions into the detector.

21. The method of claim 20, wherein a rate of product ion ejection events is greater than that of precursor m/z fragmentation events and is timed to preserve a relationship of product ions to their respective precursor ions.

22. The method of claim 21, wherein the product ion m/z information is determined by a temporal signal detected at a given product ion ejection event while the precursor m/z information is deduced from a time at which that signal is detected within one full mass scan.
